(19) United States
(12) Patent Application Publication
(10) Pub. No.: US 2024/0177865 A1
(43) Pub. Date: May 30, 2024

(54) APPARATUS AND METHOD OF PREDICTING PRESSURE ULCERS

(71) Applicant: ELECTRONICS AND TELECOMMUNICATIONS RESEARCH INSTITUTE, Daejeon (KR)

(72) Inventors: Jeong Eun KIM, Daejeon (KR); Seihyoung LEE, Daejeon (KR); Hyun Seo KANG, Daejeon (KR); Gi Hyeon MIN, Daejeon (KR); Kwang Su YUN, Daejeon (KR); Kwon-Seob LIM, Daejeon (KR)

(21) Appl. No.: 18/339,453

(22) Filed: Jun. 22, 2023

(30) Foreign Application Priority Data
Nov. 30, 2022 (KR) 10-2022-0164385

(51) Int. Cl. G16H 50/30; A61B 5/00; A61B 5/026

(52) U.S. Cl. CPC G16H 50/30; A61B 5/0073; A61B 5/026; A61B 5/447; A61B 5/7275

(57) ABSTRACT

A method of predicting pressure ulcers is provided. The method includes predicting, by a first pressure ulcer predictor, occurrence of pressure ulcers of a patient to output first prediction result data, based on body data of the patient, predicting, by a second pressure ulcer predictor, occurrence of pressure ulcers of the patient to output second prediction result data, based on whole body pressure data of the patient, predicting, by a third pressure ulcer predictor, occurrence of pressure ulcers of the patient to output third prediction result data, based on skin image data of the patient, and concatenating the first to third prediction result data to output final prediction result data.

### CROSS-REFERENCE TO RELATED APPLICATIONS

This application claims the benefit of the Korean Patent Application No. 10-2022-0164385 filed on Nov. 30, 2022, which is hereby incorporated by reference as if fully set forth herein.

### BACKGROUND

### Field of the Invention

The present invention relates to technology for predicting the occurrence of pressure ulcers, and more particularly, to technology which may analyze, by using artificial intelligence (AI), time-space pressure data of a patient collected in real time and may predict the occurrence of pressure ulcers on the basis of a result of the analysis.

### Discussion of the Related Art

Pressure ulcers denote the damage of skin tissue which occurs due to pressure, a friction, or a shearing force continuously applied to a body part when a person is continuously sitting or lying in one posture.

Recently, various researches for preventing the occurrence of pressure ulcers. For example, there are pressure ulcer risk assessment tools. Representative examples of the pressure ulcer risk assessment tools include Braden scale.

The Braden scale classifies a pressure ulcer occurrence risk into a low risk, a medium risk, a high risk, and a super-high risk by using a method which defines several items associated with a pressure ulcer risk factor and where a user (a doctor, a nurse, or the like) directly records assessment scores for each item.

However, because such a method is strong in dependence on the experience or subjective view of a user, a deviation of assessment results of assessors is very large. Due to this, there is a problem in accuracy and reliability.

### SUMMARY

An aspect of the present invention is directed to providing an apparatus and a method, which may overall analyze pressure ulcer risk score data measured by using a pressure ulcer risk assessment tool, whole body pressure data obtained by using a pressure sensor installed in a matrix of a patient, and skin image data obtained by photographing a pressure ulcer region of the patient with a camera, thereby predicting the occurrence of pressure ulcers.

Another aspect of the present invention is directed to providing an apparatus and a method, which may provide a medical team with a three-dimensional (3D) image or an augmented reality (AR) image obtained by synthesizing a three-dimensionally modeled patient with body position data (or body posture data), which is obtained in a process of overall analyzing the pressure ulcer risk score data, the whole body pressure data, and the skin image data to predict the occurrence of pressure ulcers, and pressure distribution data which is changed over time.

To achieve these and other advantages and in accordance with the purpose of the invention, as embodied and broadly described herein, there is provided a method of predicting pressure ulcers, performed by a processor included in a computing device, the method including: predicting, by a first pressure ulcer predictor, occurrence of pressure ulcers of a patient to output first prediction result data, based on body data of the patient; predicting, by a second pressure ulcer predictor, occurrence of pressure ulcers of the patient to output second prediction result data, based on whole body pressure data of the patient; predicting, by a third pressure ulcer predictor, occurrence of pressure ulcers of the patient to output third prediction result data, based on skin image data of the patient; and concatenating the first to third prediction result data to output final prediction result data.

In an embodiment, the body data may include pressure ulcer risk score data measured by a pressure ulcer risk assessment tool, micro blood flow measurement data measured by a micro blood flow measurement device, oxygen partial pressure measurement data measured by an oxygen partial pressure measurement device, and personal health record data.

In an embodiment, the skin image data may include terahertz image data, skin tomography image data, and general skin image data obtained by photographing skin with a general camera.

In an embodiment, the outputting of the first prediction result data may include outputting the first prediction result data by using an artificial intelligence model including at least one of support vector machine (SVM) and k-nearest neighbor (KNN).

In an embodiment, the outputting of the second prediction result data may include outputting the second prediction result data by using an artificial intelligence model including recurrent neural network (RNN) and long short-term memory model (LSTM).

In an embodiment, the outputting of the third prediction result data may include outputting the third prediction result data by using an artificial intelligence model including at least one of convolutional neural network (CNN), support vector machine (SVM), and k-nearest neighbor (KNN).

In an embodiment, the first to third prediction result data may be values representing a pressure ulcer incidence by percentage units, and the outputting of the final prediction result data may include: summating pressure ulcer incidences respectively corresponding to the first to third prediction result data, for concatenating the first to third prediction result data; and outputting a value, obtained by summating the pressure ulcer incidences, as the final prediction result data.

In an embodiment, the outputting of the second prediction result data may include: converting the whole body pressure data into a pressure distribution image; detecting a key point, corresponding to a pressure ulcer region of the patient, from the pressure distribution image; tracking the detected key point and position movement of the detected key point to detect body posture data of the patient; calculating average pressure data, representing an average value of pressure values distributed in a region including the detected key point, by certain time units; and outputting the second prediction result data, based on the body posture data and the average pressure data changed by certain time units.

In an embodiment, the region may be a circular region having a certain radius with respect to the detected key point.

In another aspect of the present invention, there is provided a computing device for predicting pressure ulcers, the computing device including: a first pressure ulcer predictor configured to analyze occurrence of pressure ulcers of a patient to output first prediction result data, based on body data of the patient; a second pressure ulcer predictor configured to analyze occurrence of pressure ulcers of the patient to output second prediction result data, based on whole body pressure data of the patient; a third pressure ulcer predictor configured to analyze occurrence of pressure ulcers of the patient to output third prediction result data, based on skin image data of the patient; and a data concatenation unit configured to concatenate the first to third prediction result data to output final prediction result data.

In an embodiment, the first to third prediction result data may be values representing a pressure ulcer incidence by percentage units, and the data concatenation unit may output, as the final prediction result data, a value obtained by summating pressure ulcer incidences respectively corresponding to the first to third prediction result data.

In an embodiment, the data concatenation unit may apply different weight values to the pressure ulcer incidences respectively corresponding to the first to third prediction result data to calculate the final prediction result data.

In an embodiment, the data concatenation unit may apply a highest weight value to the second prediction result data predicted based on the whole body pressure data of the patient.

In an embodiment, the second pressure ulcer predictor may include: an image processor configured to convert the whole body pressure data into a pressure distribution image; a key point detector configured to detect a key point, corresponding to a pressure ulcer region of the patient, from the pressure distribution image and calculate average pressure data, representing an average value of pressure values distributed in a region including the detected key point, by certain time units; and an artificial intelligence configured to output the second prediction result data, based on the average pressure data changed by certain time units.

In an embodiment, the key point detector may track the detected key point and position movement of the detected key point to detect body posture data of the patient, and the artificial intelligence model may output the second prediction result data, based on the body posture data and the average pressure data.

It is to be understood that both the foregoing general description and the following detailed description of the present invention are exemplary and explanatory and are intended to provide further explanation of the invention as claimed.

### BRIEF DESCRIPTION OF THE DRAWINGS

FIG. 1 is a flowchart for describing a process of predicting the occurrence of pressure ulcers of a patient, according to an embodiment of the present invention.

FIG. 2 is a diagram for describing an example of a pressure distribution image and a labeled key point(s) detected from the pressure distribution image, according to an embodiment of the present invention.

FIG. 3 is a diagram schematically illustrating a database including average pressure data calculated at a certain time interval, according to an embodiment of the present invention.

FIG. 4 is a diagram for describing a region set with respect to a key point corresponding to sacrum in FIG. 3.

FIG. 5 is a diagram for describing an example of a graphical user interface (GUI) screen configured based on performing of step S420 illustrated in FIG. 1.

FIG. 6 is a configuration diagram of a computing device for predicting the occurrence of pressure ulcers of a patient, according to an embodiment of the present invention.

### DETAILED DESCRIPTION OF THE INVENTION

Hereinafter, example embodiments of the invention will be described in detail with reference to the accompanying drawings. In describing the invention, to facilitate the entire understanding of the invention, like numbers refer to like elements throughout the description of the figures, and a repetitive description on the same element is not provided.

In the following description, the technical terms are used only for explain a specific exemplary embodiment while not limiting the present invention. The terms of a singular form may include plural forms unless referred to the contrary. The meaning of ‘comprise’, ‘include’, or ‘have’ specifies a property, a region, a fixed number, a step, a process, an element and/or a component but does not exclude other properties, regions, fixed numbers, steps, processes, elements and/or components.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present invention may analyze time-space pressure data in real time by using AI, predict the occurrence of pressure ulcers on the basis of a result of the analysis, and provide a medical team with a result of the prediction, thereby preventing the occurrence of pressure ulcers.

Moreover, the present invention may track positions of key points which are high in probability of occurrence of pressure ulcers, in addition to estimating a posture of a patient over time, and may calculate a duration time and a pressure distribution applied to a region including the key point, thereby enhancing the accuracy of prediction of occurrence of pressure ulcers.

Hereinafter, embodiments of the present invention will be described in detail with reference to the accompanying drawings.

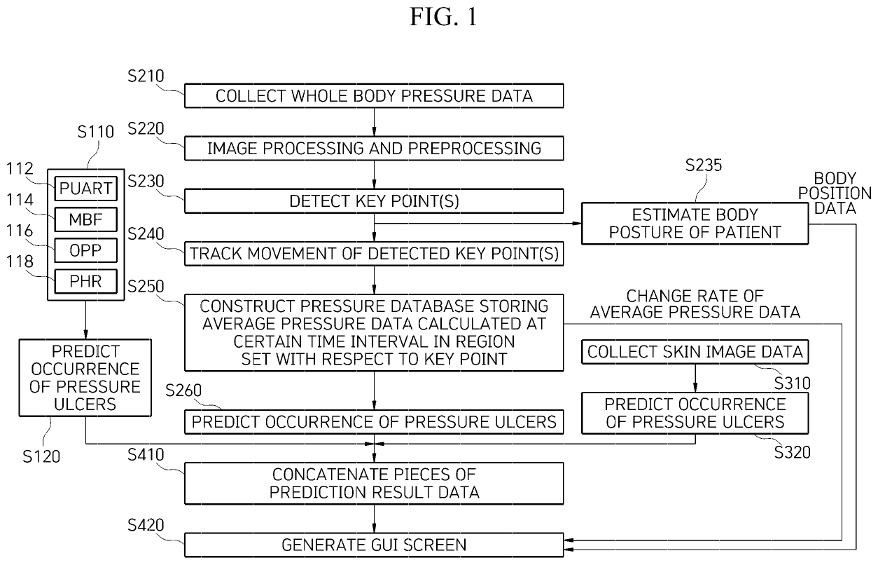

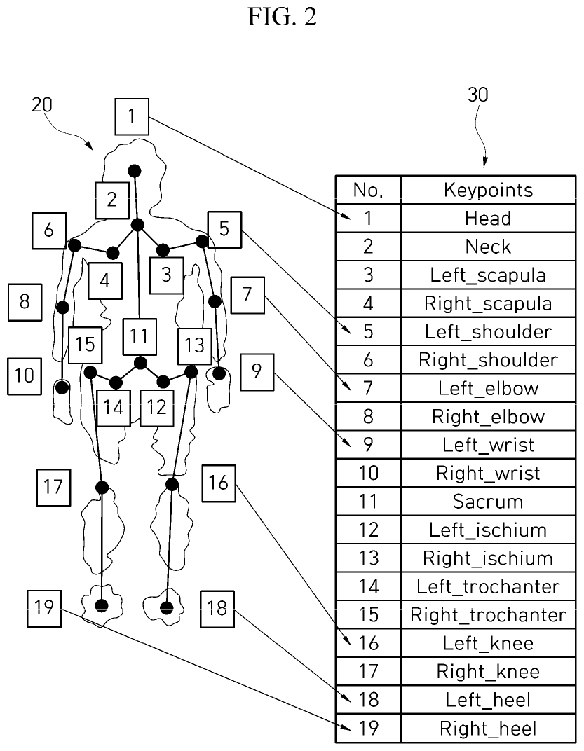

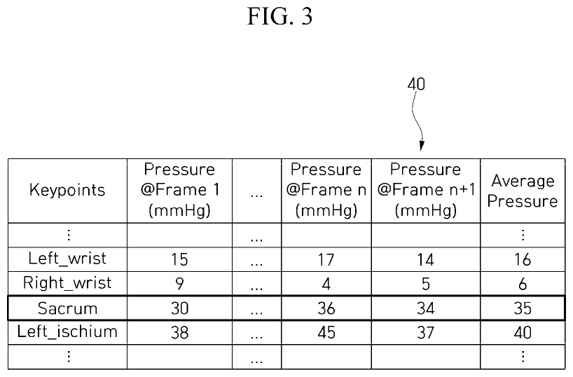

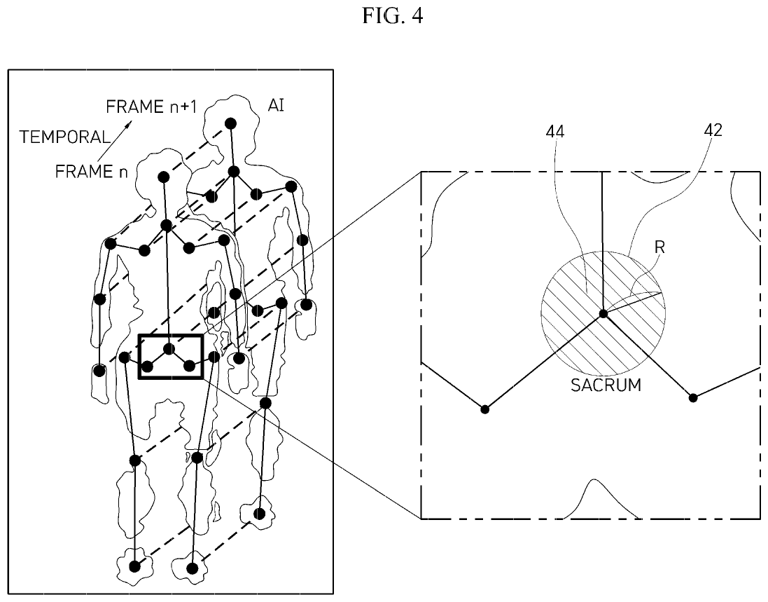

FIG. 1 is a flowchart for describing a process of predicting the occurrence of pressure ulcers of a patient, according to an embodiment of the present invention. FIG. 2 is a diagram for describing an example of a pressure distribution image and a labeled key point(s) detected from the pressure distribution image, according to an embodiment of the present invention. FIG. 3 is a diagram schematically illustrating a database including average pressure data calculated at a certain time interval, according to an embodiment of the present invention. FIG. 4 is a diagram for describing a region set with respect to a key point corresponding to sacrum in FIG. 3.

First, referring to FIG. 1, steps S110, S120, S210 to S260, S310, S320, S410, and S420 may be performed in a computing system implemented to include a processor, a memory, an input interface, an output interface, a storage device, and a communication device.

The computing system according to various embodiments of the present invention may include, for example, at least one of a smartphone, a tablet personal computer (PC), a desktop PC, a laptop PC, a netbook PC, a workstation, a server, a personal digital assistant (PDA), a portable multimedia player (PMP), an MP3 player, a camera, and a wearable device.

In another embodiment, the computing system may include, for example, at least one of various medical devices (for example, mobile medical devices (for example, a blood glucose monitoring device, a heartbeat measuring device, a blood pressure measuring device, and a body temperature measuring device), magnetic resonance angiography (MRA) device, a magnetic resonance imaging (MRI) device, computed tomography (CT) device, an imaging device, or an ultrasonic device), a navigation device, a global positioning system (GPS) receiver, an event data recorder (EDR), a flight data recorder (FDR), an automotive infotainment device, a naval electronic device (e.g., naval navigation device, gyroscope, or compass), an avionic electronic device, a security device, an industrial or consumer robot, an automation teller's machine (ATM), a point of sales (POS), and an Internet of things (IOT) device.

In another embodiment, the computing system may be flexible, or may be a combination of two or more of various devices described above. The computing system according to an embodiment of the present invention is not limited to the devices described above.

In FIG. 1, steps S110 and S120, steps S210 to S260, and steps S310 and S320 may be simultaneously performed in parallel. Also, steps S110 and S120, steps S210 to S260, and steps S310 and S320 may be selectively performed. For example, steps S210 to S260 and steps S310 and S320 may be simultaneously performed in parallel, and steps S110 and S120 may be omitted. Also, steps S110 and S120 and steps S210 to S260 may be simultaneously performed in parallel, and steps S310 and S320 may be omitted. Also, steps S210 to S260 may be simultaneously performed in parallel, and steps S110 and S120 and steps S310 and S320 may be omitted. In a case where steps S210 to S260 are independently performed, a process of concatenating pieces of prediction result data performed in step S410 described below may be omitted. Also, steps S220 to S260 may be integrated into one step.

S110 and S120

First, in step S110, a process of collecting pressure ulcer risk score data 112 measured by a pressure ulcer risk assessment tool (PURAT), micro blood flow measurement data 114 measured by a micro blood flow measurement device (MBF), oxygen partial pressure measurement data 116 measured by an oxygen partial pressure measurement device (OPP), and personal health record data 118 may be performed. Here, the PURAT may include Braden scale, Norton scale, and Waterlow scale. The personal health record data 118 may be obtained from a hospital server connected with a computing system of a medical team. The personal health record data 118 may include, for example, blood test data, age, sex, weight, and a medical history.

Subsequently, in step S120, a process of predicting, by an AI model, the occurrence of pressure ulcers by using the pressure ulcer risk score data 112, the micro blood flow measurement data 114, the oxygen partial pressure measurement data 116, and the personal health record data 118 may be performed. Here, the AI model may be a model which is previously learned based on supervised learning, unsupervised learning, or a combination thereof so as to predict pieces of learning data having the same attributes as those of the pressure ulcer risk score data 112, the micro blood flow measurement data 114, the oxygen partial pressure measurement data 116, and the personal health record data 118, and for example, may be implemented as a machine learning algorithm such as support vector machine (SVM) or k-nearest neighbor (KNN).

S210 to S260

First, in step S210, a process of collecting in real time whole body pressure data of pressure, applied to a whole body of a patient, from a plurality of pressure sensors installed in a matrix of the patient may be performed. The whole body pressure data may include a pressure value measured by each pressure sensor, and the pressure value may denote pressure intensity applied to a body of the patient.

Subsequently, in step S220, image processing and preprocessing processes on the whole body pressure data may be performed sequentially, in parallel, or selectively. The image processing process may denote a process of converting whole body pressure data, collected in real time, into frame-unit pressure distribution image data (hereinafter referred to as a pressure distribution image). Here, the pressure distribution image may include a two-dimensional (2D) image where a distribution of pressure values included in the whole body pressure data is divided by colors like a thermal image. The preprocessing process may include a process of refining the pressure distribution image, a noise removal process, and a resolution control process. The present invention may not be characterized in the image processing and preprocessing processes, and thus, a description thereof may be replaced with known technology.

Subsequently, in step S230, the AI model may perform a process of detecting a key point(s) corresponding to a pressure ulcer-prone region (a pressure ulcer occurrence point) of the patient from a preprocessed pressure distribution image may be performed. Here, the key point may be selected based on the pressure ulcer-prone region. The AI model for detecting the key point(s) may label pressure values, included in learning whole body pressure body, to learning key points, and then, may be constructed through pre-learning performed based on supervised learning, unsupervised learning, or a combination thereof by using labeled learning key points and/or learning whole body pressure data. In FIG. 2, an example of the pressure distribution image 20 and the labeled key points 30 detected from the pressure distribution image 20 is illustrated. The AI model for detecting a key point(s) may be implemented with, for example, a convolutional neural network (CNN) which is widely used in image processing.

Subsequently or simultaneously, in step S235, a process of estimating a body posture (a body position) of the patient on the basis of the detected key point(s) may be performed. Such a body posture estimation process may be performed by the AI model for detecting the key point(s).

Subsequently, in step S240, a process of tracking the position movement of the key point(s) based on the movement of the patient. The body position of the patient may be changed over time, and thus, the key point(s) detected in step S230 may move. The position movement of the key point(s) may be tracked based on an optical flow scheme or a visual object tracking scheme. For example, the position movement of the key point(s) may be calculated based on a difference value between a position of a key point(s) of a previous frame (an n^{th} frame) and a position of a key point(s) of a current frame (an n+1^{st} frame).

Subsequently, in step S250, a process of constructing a pressure database storing average pressure data calculated at a certain time interval in a region set with respect to each of the tracked key points may be performed. Here, the average pressure data may denote an average value of pressure values distributed in the region. In FIG. 3, an example of the pressure database storing the average pressure data calculated at the certain time interval is illustrated. In FIG. 4, a region set with respect to a key point corresponding to sacrum in FIG. 3 is illustrated. As illustrated in FIG. 4, a region set with respect to the tracked key point 44 may be, for example, a circular region having a radius R. The constructed pressure database may be time-serial data of pressure by key points, and thus, may have information capable of knowing a time for which certain pressure is retained in each key point.

Subsequently, in step S260, a process of predicting, by the AI model, the occurrence of pressure ulcers by using the average pressure data calculated at the certain time interval (i.e., the average pressure data changed at the certain time interval) and stored in the database (40 of FIG. 3) may be performed. Here, body position data (or body posture data) representing a posture of a patient obtained in step S235 may be further used as input data, so as to predict the occurrence of pressure ulcers. The body position data may be used for determining a body position change time of the patient. The AI model may be a model which is previously learned based on supervised learning, unsupervised learning, or a combination thereof so as to predict the occurrence of pressure ulcers, based on the body position data and/or the average pressure data calculated at the certain time interval, and for example, may be implemented as a sequence learning model suitable for time-serial data processing, like recurrent neural network (RNN) or long short-term memory model (LSTM) which is a type of RNN.

S310 and S320

A skin state of a patient may act as a main cause of pressure ulcers. Therefore, in an embodiment of the present invention, data associated with a skin state of a patient may be used as data for improving the accuracy of prediction of pressure ulcers.

First, in step S310, a process of collecting skin image data (hereinafter referred to as a skin image) may be performed. The skin image may be, for example, an image expressing a skin state and may include a terahertz image which enables a moisture state of a skin to be determined, a skin tomography image which is capable of being seen without cutting skin, and a general skin image which is obtained by photographing skin with a general camera. Here, the terahertz image may be obtained by a terahertz wave device, and the skin tomography image may be obtained by an optical coherence tomography (OCT) device.

Subsequently, in step S320, a process of predicting the occurrence of pressure ulcers by using the skin image data as input data may be performed. Here, the AI model may be a model which is previously learned based on supervised learning, unsupervised learning, or a combination thereof so as to predict the occurrence of pressure ulcers, based on the skin image data, and for example, may be implemented as CNN, SVM, and KNN.

S410 and S420

First, in step S410, when a prediction result (hereinafter referred to as first prediction result data) based on performing of step S120, a prediction result (hereinafter referred to as second prediction result data) based on performing of step S260, and a prediction result (hereinafter referred to as third prediction result data) based on performing of step S320 are obtained, a process of concatenating the first to third prediction result data may be performed. Here, each of the first to third prediction result data, for example, may be a value where a pressure ulcer incidence of a patient is represented by a percentage (%) unit. In this case, concatenation may be an operation of summating pressure ulcer incidences respectively obtained based on performing of steps S120, S260, and S320. In this case, a weight value may be applied. For example, because whole body pressure data acts as a direct cause of pressure ulcers, a pressure ulcer incidence based on performing of step S260 may be multiplied by a highest weight value, a pressure ulcer incidence based on performing of step S120 may be multiplied by a lowest weight value, and a pressure ulcer incidence based on performing of step S320 may be multiplied by a medium weight value. Subsequently, final prediction result data of a pressure ulcer incidence may be obtained by summating pressure ulcer incidences to which different weight values are applied. Also, the first to third prediction result data may be selectively concatenated. For example, the second prediction result data (S260) and the third prediction result data (S320) may be concatenated, and the first prediction result data (S120) may be excluded from concatenation. Alternatively, the first prediction result data (S120) and the second prediction result data (S260) may be concatenated, and the third prediction result data (S320) may be excluded from concatenation. Alternatively, the second prediction result data (S260) may be used as the final prediction result data.

Subsequently, a process of generating a GUI screen provided to a medical team on the basis of the final prediction result data may be performed.

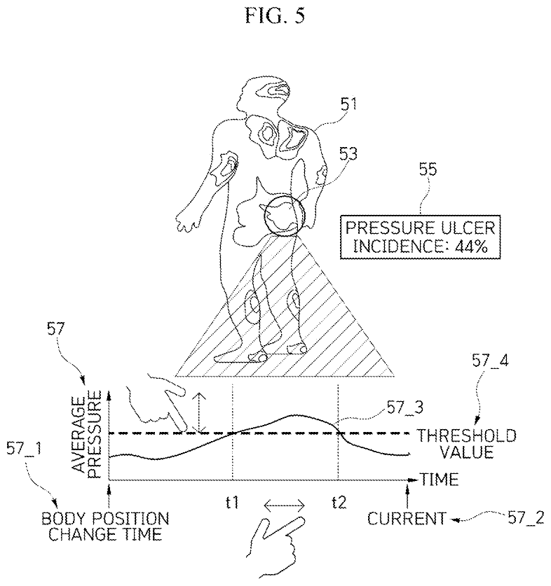

FIG. 5 is a diagram for describing an example of a GUI screen configured based on performing of step S420 illustrated in FIG. 1.

Referring to FIG. 5, the GUI screen may be generated by a GUI program for a medical team (a manager). The GUI program may generate the GUI screen by using a change rate of each of final prediction result data (for example, an incidence (%)) of the occurrence of pressure ulcers obtained based on performing of step S410, body position data (or body posture data) obtained based on performing of step S235, and average pressure data changed over time and obtained from a pressure database (for example, 30 of FIG. 3) constructed based on performing of step S250.

The GUI screen, as illustrated in FIG. 5, may include a patient avatar 51 which is modeled as a 3D image. In this case, pressure distribution data divided by colors may be synthesized with the patient avatar 51. The pressure distribution data may be obtained by processing pressure values included in the whole body pressure data which is collected in real time in step S210 or average pressure data obtained by processing the average pressure data stored by key points from a pressure database (for example, 40 of FIG. 3). A patient object 51 may be a real patient image, and in this case, a medical team may recognize the real patient image synthesized with the pressure distribution data through AR.

A pressure ulcer occurrence region 53 representing a region 42 set with respect to a key point may be disposed in the patient avatar 51 or the real patient image, and a pressure ulcer incidence 55 constructed by a combination of a text and a digit may be displayed at an adjacent position of the pressure ulcer occurrence region 53.

Moreover, the GUI screen may include a graph object 57 where a change rate of average pressure data up to a current time 57_2 from a body position change time 57_1 is represented by a curve 57_3. The body position change time 57_1 may be obtained from body position data.

Moreover, the GUI screen may be configured to have a touch function. For example, when the medical team touches a specific body part of the patient avatar 51, the graph object 57 may be disposed in the patient avatar 51, and average pressure data exceeding a threshold value 57_4 in a specific time zone t1˜t2 may be checked through the displayed graph object 57.

Moreover, the GUI screen may be configured to provide an alarm function. The threshold value 57_4 may be set by upward and downward performing dragging in a state where a finger touches a line (or a dotted line) representing the threshold value 57_4, or the specific time zone t1˜t2 may be set by widening or narrowing the specific time zone t1˜t2 with a finger, and then, when the curve 57_3 representing a change rate of average pressure data measured in real time is greater than the threshold value 57_4 in the specific time zone t1˜t2, an alarm is set to be sounded. As described above, a medical team may efficiently manage the pressure ulcers of a patient through a GUI screen.

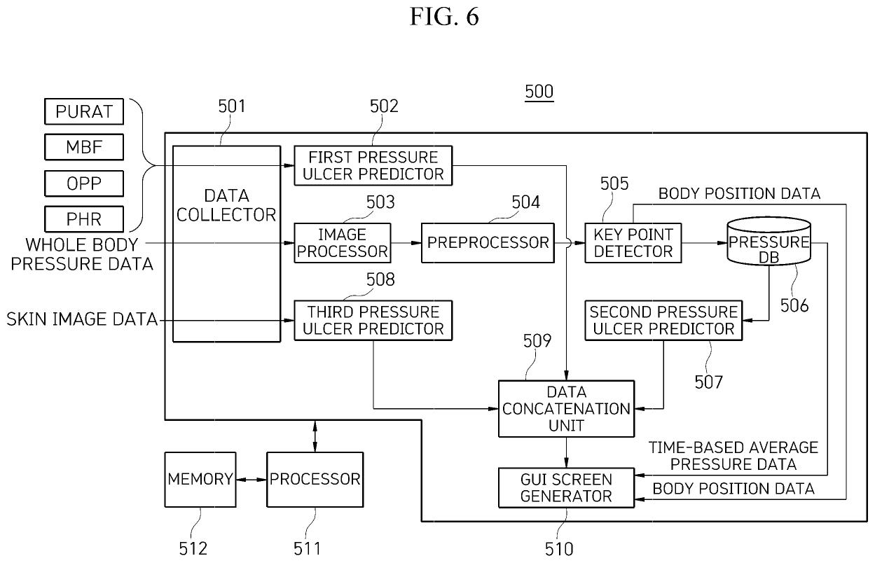

FIG. 6 is a configuration diagram of a computing device 500 for predicting the occurrence of pressure ulcers of a patient, according to an embodiment of the present invention.

Referring to FIG. 6, the computing device 500 according to an embodiment of the present invention may include a data collector 501, a first pressure ulcer predictor 502, an image processor 503, a preprocessor 504, a key point detector 505, a pressure database (or a storage medium) 506, a second pressure ulcer predictor 507, a third pressure ulcer predictor 508, a data concatenation unit 509, a GUI screen generator 510, a processor 511, and a memory 512.

The data collector 510 may be an element which processes steps S110, S210, and S310 illustrated in FIG. 1. That is, the data collector 501 may collect the pressure ulcer risk score data 112 measured by the PURAT, the micro blood flow measurement data 114 measured by the MBF, the oxygen partial pressure measurement data 116 measured by the OPP, and the personal health record data 118. Also, the data collector 510 may collect whole body pressure data from a plurality of pressure sensors installed in a matrix of a patient. Also, the data collector 510 may collect skin image data. Here, the skin image data may include a terahertz image representing a moisture state of skin, a skin tomography image which is capable of being seen without cutting skin, and a general skin image which is obtained by photographing skin with a general camera.

The term “body data” described in the claims may be used as the term including the pressure ulcer risk score data 112, the micro blood flow measurement data 114, the oxygen partial pressure measurement data 116, and the personal health record data 118.

Although not shown, the data collector 501 may include an input interface, a communication interface, and a storage medium. A user may input the pressure ulcer risk score data, the micro blood flow measurement data, the oxygen partial pressure measurement data, the personal health record data, whole body pressure data, and skin image data to the computing device 50 by using the input interface. Also, the computing device 50 may receive the micro blood flow measurement data, the oxygen partial pressure measurement data, the personal health record data, the whole body pressure data, and the skin image data through the communication interface which supports wired or wireless communication. Here, the wireless communication may include short-range wireless communication such as WiFi or Bluetooth, Internet wireless communication, and mobile communication (for example, 3G, 4G, 5G, and 6G).

The first pressure ulcer predictor 502 may be an element which processes steps S110, S210, and S310 illustrated in FIG. 1 and may be a software module including an AI model which is previously learned to predict the occurrence of pressure ulcers of a patient by using the pressure ulcer risk score data, the micro blood flow measurement data, the oxygen partial pressure measurement data, and the personal health record data as input data.

The image processor 503 and the preprocessor 504 may process step S220 illustrated in FIG. 1. The image processor 503 may convert the whole body pressure data, collected in real time, into frame-unit pressure distribution image data by using a known image processing technique. In FIG. 6, each of the image processor 503 and the preprocessor 504 is illustrated as an independent element, but is not limited thereto and may be integrated as one element. Also, in an embodiment of the present invention, the preprocessor 504 may be omitted based on the hardware performance of the computing device 500.

The key point detector 505 may be an element which processes steps S230, S235, and S240 illustrated in FIG. 1 and may be a software module including an AI model which is previously learned to detect a key point, estimate a posture (a body position) of the patient based on the detected key point, and estimate the position movement of the detected key point.

The key point detector 505 may detect a key point corresponding to a pressure ulcer region of the patient from the pressure distribution image and may calculate, by certain time units, average pressure data representing an average value of pressure values distributed in a region including the detected key point. Also, the key point detector 505 may track the detected key point and the position movement of the detected key point to detect body position data of the patient.

The storage medium 506 may be a device which stores a pressure database constructed with average pressure data calculated by certain time units in a region with respect to a key point, based on performing of step S250 illustrated in FIG. 1.

The second pressure ulcer predictor 507 may be a software module including an AI model which is previously learned to predict the occurrence of pressure ulcers by using the average pressure data, stored in the pressure database and calculated by certain time units, as input data.

The third pressure ulcer predictor 508 may be a software module including an AI model which is previously learned to predict the occurrence of pressure ulcers by using the skin image data as input data.

The data concatenation unit 509 may concatenate pieces of prediction result data of the first to third pressure ulcer predictors 502, 507, and 508 to calculate final prediction result data. Each of the pieces of prediction result data may be, for example, a value obtained by digitizing a pressure ulcer incidence of the patient by percentage (%) units. In this case, data concatenation may denote an operation of summating or multiplying pressure ulcer incidences (%) which are the prediction results of the first to third pressure ulcer predictors 502, 507, and 508.

For example, when a first pressure ulcer incidence predicted by the first pressure ulcer predictor 502 is A %, a second pressure ulcer incidence predicted by the second pressure ulcer predictor 507 is B %, and a third pressure ulcer incidence predicted by the third pressure ulcer predictor 508 is C %, the final prediction result data may be “A %+B %+C %”.

As another example, the data concatenation unit 509 may apply different weight values to the pressure ulcer incidences to calculate the final prediction result data. For example, when a weight value w₁ is applied to the first pressure ulcer incidence, a weight value w₂ is applied to the first pressure ulcer incidence, and a weight value w₃ is applied to the first pressure ulcer incidence, the final prediction result data may be “(w₁×A) %+(w₂×B) %+(w₃×C) %”. In this case, a highest weight value may be applied to a result obtained by predicting the occurrence of pressure ulcers from data (for example, the whole body pressure data) which is the most reliable in predicting the occurrence of pressure ulcers. On the other hand, a lowest weight value may be applied to a result obtained by predicting the occurrence of pressure ulcers from data (for example, input data including the pressure ulcer risk score data, the micro blood flow measurement data, the oxygen partial pressure measurement data, and the personal health record data) which is relatively low in reliability, in predicting the occurrence of pressure ulcers.

The GUI screen generator 510 may generate a patient-based customized GUI screen, based on the final prediction result data input from the data concatenation unit 509, the body position data input from the key point detector 505, and the time-based average pressure data input from the pressure database. A configuration of the GUI screen has been described in detail with reference to FIG. 5, and thus, a description thereof may be omitted. The GUI screen generator 510 may be a graphics processor unit (GPU), or may be a hardware module included in the GPU.

In FIG. 6, each of the first to third pressure ulcer predictors 502, 507, and 508 is illustrated as an independent element, but is not limited thereto and may be integrated as one element (i.e., one AI model).

The processor 511 may control and manage overall operations of the elements 501 to 510 described above. The processor 720 may be implemented as, for example, at least one central processing unit (CPU), at least one GPU, at least one application processor, at least one system on chip (SoC), or at least one micro controller unit (MCU). Also, the processor 511 may be a semiconductor device which executes instructions stored in the memory 512 or a storage device. Also, the processor 511 may control learning (for example, supervised learning, unsupervised learning, etc.) of the AI models included in the first to third pressure ulcer predictors 502, 507, and 508.

Moreover, although not shown in FIG. 6, the computing device 500 may further include a display device which displays a GUI screen generated by the GUI screen generator 510. The display device may include a touch panel to have a touch function.

Moreover, in FIG. 6, the image processor 503, the preprocessor 504, the key point detector 505, the pressure database 506, and the second pressure ulcer predictor 507 are illustrated as independent elements, but are not limited thereto and may be integrated into one element. For example, the second pressure ulcer predictor 507 may include the image processor 503, the preprocessor 504, the key point detector 505, and the pressure database 506.

Moreover, in FIG. 6, the first pressure ulcer predictor 502, the third pressure ulcer predictor 508, and the data concatenation unit 509 may be omitted. In this case, the computing device 500 may include the data collector 501, the image processor 503, the preprocessor 504, the key point detector 505, the pressure database 506, the second pressure ulcer predictor 507, and the GUI screen generator 510, the processor 511, and the memory 512 and may not include the first pressure ulcer predictor 502, the third pressure ulcer predictor 508, and the data concatenation unit 509.

An embodiment of the present invention may be implemented as a method implemented in a computer, or may be implemented as a non-transitory computer-readable medium storing a computer-executable instruction. In an embodiment, when executed by the processor, the computer-readable instruction may perform a method according to at least one aspect of the present invention. Also, the method according to an embodiment of the present invention may be implemented as a program instruction type capable of being performed by various computer means and may be stored in a computer-readable recording medium. The computer-readable recording medium may include a program instruction, a data file, or a data structure, or a combination thereof. The program instruction recorded in the computer-readable recording medium may be specially designed for an embodiment of the present invention, or may be known to those skilled in the computer software art and may be used. The computer-readable recording medium may store may include a hardware device which stores and executes the program instruction. For example, the computer-readable recording medium may be a magnetic media such as a hard disk, a floppy disk, and a magnetic tape, an optical media such as CD-ROM or DVD, a magneto-optical media such as a floptical disk, ROM, RAM, or flash memory. The program instruction may include a high-level language code executable by a computer such as an interpreter, in addition to a machine language code such as being generated by a compiler.

According to the embodiments of the present invention, the occurrence of pressure ulcers may be predicted by overall analyzing pressure ulcer risk score data measured by using a pressure ulcer risk assessment tool, whole body pressure data of a patient, and skin-related image data, and thus, accuracy and reliability may be more enhanced than a conventional method which predicts the occurrence of pressure ulcers by using a pressure ulcer risk assessment tool.

Moreover, the present invention may provide a medical team with a 3D image or an AR image obtained by synthesizing a three-dimensionally modeled patient object with body position data (or body posture data), which is obtained in a process of overall analyzing the pressure ulcer risk score data, the whole body pressure data, and the skin image data to predict the occurrence of pressure ulcers, and pressure distribution data which is changed over time, and thus, the medical team may intuitively and quickly check a pressure ulcer risk of an individual patient to quickly perform an appropriate action.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A method of predicting pressure ulcers, performed by a processor included in a computing device, the method comprising:
predicting, by a first pressure ulcer predictor, occurrence of pressure ulcers of a patient to output first prediction result data, based on body data of the patient;
predicting, by a second pressure ulcer predictor, occurrence of pressure ulcers of the patient to output second prediction result data, based on whole body pressure data of the patient;
predicting, by a third pressure ulcer predictor, occurrence of pressure ulcers of the patient to output third prediction result data, based on skin image data of the patient; and
concatenating the first to third prediction result data to output final prediction result data.

2. The method of claim 1, wherein the body data comprises pressure ulcer risk score data measured by a pressure ulcer risk assessment tool, micro blood flow measurement data measured by a micro blood flow measurement device, oxygen partial pressure measurement data measured by an oxygen partial pressure measurement device, and personal health record data.

3. The method of claim 1, wherein the skin image data comprises terahertz image data, skin tomography image data, and general skin image data obtained by photographing skin with a general camera.

4. The method of claim 1, wherein the outputting of the first prediction result data comprises outputting the first prediction result data by using an artificial intelligence model including at least one of support vector machine (SVM) and k-nearest neighbor (KNN).

5. The method of claim 1, wherein the outputting of the second prediction result data comprises outputting the second prediction result data by using an artificial intelligence model including recurrent neural network (RNN) and long short-term memory model (LSTM).

6. The method of claim 1, wherein the outputting of the third prediction result data comprises outputting the third prediction result data by using an artificial intelligence model including at least one of convolutional neural network (CNN), support vector machine (SVM), and k-nearest neighbor (KNN).

7. The method of claim 1, wherein the first to third prediction result data are values representing a pressure ulcer incidence by percentage units, and
the outputting of the final prediction result data comprises:
summating pressure ulcer incidences respectively corresponding to the first to third prediction result data, for concatenating the first to third prediction result data; and
outputting a value, obtained by summating the pressure ulcer incidences, as the final prediction result data.

8. The method of claim 1, wherein the outputting of the second prediction result data comprises:
converting the whole body pressure data into a pressure distribution image;
detecting a key point, corresponding to a pressure ulcer region of the patient, from the pressure distribution image;
tracking the detected key point and position movement of the detected key point to detect body posture data of the patient;
calculating average pressure data, representing an average value of pressure values distributed in a region including the detected key point, by certain time units; and
outputting the second prediction result data, based on the body posture data and the average pressure data changed by certain time units.

9. The method of claim 8, wherein the region is a circular region having a certain radius with respect to the detected key point.

10. A computing device for predicting pressure ulcers, the computing device comprising:
a first pressure ulcer predictor configured to analyze occurrence of pressure ulcers of a patient to output first prediction result data, based on body data of the patient;
a second pressure ulcer predictor configured to analyze occurrence of pressure ulcers of the patient to output second prediction result data, based on whole body pressure data of the patient;
a third pressure ulcer predictor configured to analyze occurrence of pressure ulcers of the patient to output third prediction result data, based on skin image data of the patient; and
a data concatenation unit configured to concatenate the first to third prediction result data to output final prediction result data.

11. The computing device of claim 10, wherein the first to third prediction result data are values representing a pressure ulcer incidence by percentage units, and
the data concatenation unit outputs, as the final prediction result data, a value obtained by summating pressure ulcer incidences respectively corresponding to the first to third prediction result data.

12. The computing device of claim 11, wherein the data concatenation unit applies different weight values to the pressure ulcer incidences respectively corresponding to the first to third prediction result data to calculate the final prediction result data.

13. The computing device of claim 12, wherein the data concatenation unit applies a highest weight value to the second prediction result data predicted based on the whole body pressure data of the patient.

14. The computing device of claim 10, wherein the second pressure ulcer predictor comprises:
an image processor configured to convert the whole body pressure data into a pressure distribution image;
a key point detector configured to detect a key point, corresponding to a pressure ulcer region of the patient, from the pressure distribution image and calculate average pressure data, representing an average value of pressure values distributed in a region including the detected key point, by certain time units; and
an artificial intelligence configured to output the second prediction result data, based on the average pressure data changed by certain time units.

15. The computing device of claim 14, wherein the key point detector tracks the detected key point and position movement of the detected key point to detect body posture data of the patient, and
the artificial intelligence model outputs the second prediction result data, based on the body posture data and the average pressure data.
